SEQUENCE LISTING (3 sequences)

SEQ ID NO: 1 - DNA, 16 bp, synthetic construct
  misc_feature 1..16: note = Synthetic
  source 1..16: mol_type = other DNA
gccagccatctgttgt

SEQ ID NO: 2 - DNA, 16 bp, synthetic construct
  misc_feature 1..16: note = Synthetic
  source 1..16: mol_type = other DNA
ggagtggcaccttcca

SEQ ID NO: 3 - DNA, 21 bp, synthetic construct
  misc_feature 1..21: note = Synthetic
  source 1..21: mol_type = other DNA
tcccccttgccttccttgacc

(19) United States
(12) Patent Application Publication
(10) Pub. No.: US 2024/0209415 A1
(43) Pub. Date: Jun. 27, 2024

(54) DROPLET DIGITAL PCR ASSAY FOR DETERMINING VIRAL VECTOR GENOMIC TITER

(71) Applicant: Regeneron Pharmaceuticals, Inc., Tarrytown, NY (US)

(72) Inventors: Li Zhi, Tarrytown, NY (US); Kuan-Yu Lai, Livingston, NJ (US); Dingjiang Liu, Pleasantville, NY (US)

(21) Appl. No.: 18/529,429

(22) Filed: Dec. 5, 2023

Related U.S. Application Data
(60) Provisional application No. 63/430,481, filed on Dec. 6, 2022.

(51) Int. Cl. C12Q 1/6806; C12Q 1/70

(52) U.S. Cl. CPC C12Q 1/6806; C12Q 1/70; C12Q 2600/166

(57) ABSTRACT

Disclosed herein are methods for quantification of viral genomes present inside viral capsids using a droplet digital PCR assay. In embodiments, the methods include preparing a sample of viral genome by sample dilution, treating the diluted sample of viral genome with DNase I/Proteinase K, analyzing the sample of viral genome by ddPCR assay, and determining a viral titer based on analysis of data obtained from the ddPCR assay. In examples, the ddPCR assay in accordance with the present disclosure produces a variability rate of no more than 5%.

### CROSS REFERENCE TO RELATED APPLICATIONS

This application claims the benefit under 35 USC § 119(e) of U.S. Provisional Application No. 63/430,481 filed Dec. 6, 2022, which is incorporated herein by reference in its entirety.

### SEQUENCE LISTING

This application incorporates by reference a computer readable Sequence Listing in ST.26 XML format, titled 11188US01_Sequence, created on Dec. 4, 2023, and containing 4,885 bytes.

### FIELD OF THE INVENTION

The present invention relates to methods for quantifying viral genomes (e.g., AAV genomes) using a droplet digital polymerase chain reaction (ddPCR) assay.

### BACKGROUND

Adeno-associated virus (AAV), which is a non-enveloped, single-stranded DNA virus, has emerged as an attractive class of therapeutic agents to deliver genetic materials to host cells for gene therapy, due to its ability to transduce a wide range of species and tissues in vivo, low risk of immunotoxicity, and mild innate and adaptive immune responses. The complex nature of viral vectors, such as AAV, require specific analytical methods to enable product testing and viral genome quantification. Recombinant AAV technology relies on proper genome packaging inside the capsids of recombinant AAV samples.

Existing techniques for droplet digital PCR (ddPCR) have enabled an expansion of DNA amplification, quantification, and diagnostic applications. However, currently available ddPCR techniques have a high rate of variability. A coefficient of variation of up to 13.44% has been found to be associated with conventional ddPCR techniques. In order to identify small differences in the titer of a gene of interest present inside the viral capsids, the high variability rate of existing ddPCR techniques poses a challenge. Thus, improved methods for sample handling are needed for accurate quantification of viral genomes present inside the viral capsids.

### BRIEF SUMMARY OF THE INVENTION

The present disclosure is directed to methods of preparing a sample of viral genome (e.g., AAV genome) and performing a droplet digital PCR assay on the prepared sample of viral genome. The current methods allow accurate quantification of viral genome by ddPCR assay. The methods of viral genome quantification, according to the present disclosure, includes sample dilution, DNase I/Proteinase K treatment, serial dilution and treatment with ddPCR master mix, and analysis of data obtained from the ddPCR assay. In exemplary embodiments, the analysis quantifies viral genomes present inside the capsids of recombinant adeno-associated viruses (rAAV) samples using primer-probes specific to recombinant viral payload. The droplet digital PCR assay in accordance with the present disclosure produces a coefficient of variation of no more than 5%.

In one aspect, the present disclosure provides a method of preparing a sample of viral genome from a sample of viral particles for a droplet digital PCR (ddPCR) assay, comprising: (a) diluting the sample of viral genome with a dilution buffer and gently mixing the diluted sample of viral genome with a relative centrifugal force ranging from about 10 to about 150 x g (all references to relative centrifugal force or RCF herein referring to a value are “x g”); (b) treating the diluted sample of viral genome with DNase I by gently mixing with a DNase I reaction mix at a relative centrifugal force of from about 10 to about 150; (c) treating the DNase I-treated sample of viral genome with Proteinase K by mixing with a Proteinase K reaction mix at a relative centrifugal force of from about 50 to about 300; (d) serially diluting the Proteinase K-treated sample of viral genome with the dilution buffer and mixing the serially diluted sample of viral genome with a relative centrifugal force of from about 50 to about 300; (e) preparing a master mix comprising a primer-probe mix for the ddPCR assay and mixing the prepared master mix at a relative centrifugal force of from about 50 to about 300; and (f) loading the serially diluted sample of viral genome with the master mix comprising the primer-probe mix on a ddPCR plate for the ddPCR assay.

In some embodiments, the sample of viral particles comprises adeno-associated virus (AAV) particles. In some cases, the AAV particles are of serotype AAV1, AAV2, AAV3, AAV4, AAV5, AAV6, AAV7, AAV8, AAV9, AAV-DJ, AAV-DJ/8, AAV-Rh10, AAV-retro, AAV-PHP.B, AAV8-PHP.eB, or AAV-PHP.S.

In some embodiments, the gentle mixing is conducted using a vortex mixer with a relative centrifugal force of from about 20 to about 100. In some embodiments, the gentle mixing is conducted using a vortex mixer with a relative centrifugal force of from about 40 to about 60, In some cases, the gentle mixing is performed for a period of from about 10 seconds to about 45 seconds.

In some embodiments, the mixing is carried out using a vortex mixer with a relative centrifugal force of from about 100 to about 200. In some embodiments, the mixing is carried out using a vortex mixer with a relative centrifugal force of from about 110 to about 140. In some cases, the mixing is performed for a period of from about 10 seconds to about 45 seconds.

In some embodiments, the mixing and/or gentle mixing is conducted in a tube having a tube wall with reduced nucleic acid adsorption. In some embodiments, the mixing and/or gentle mixing is performed for a period of from about 10 seconds to about 20 seconds.

In some embodiments, the serially diluted sample of viral genome is mixed with the master mix comprising the primer-probe mix at a relative centrifugal force of from about 50 to about 300 before loading on the ddPCR plate as well as after sealing of the plate. In some cases, the mixing is carried out using a vortex mixer with a relative centrifugal force of from about 100 to about 200 for 10-20 seconds.

In some embodiments, the dilution buffer comprises a non-ionic surfactant and a sheared salmon sperm DNA, and wherein the dilution buffer is filtered through a 0.20-0.25 μm syringe filter.

In some embodiments, the primer-probe mix comprises at least one oligonucleotide probe that is detectably labeled and at least two oligonucleotide primers targeting a gene of interest. In some cases, the oligonucleotide probe comprises a nucleotide sequence of SEQ ID NO: 3. In some cases, the oligonucleotide primers further comprise a forward primer with a nucleotide sequence of SEQ ID NO: 1 and a reverse primer with a nucleotide sequence of SEQ ID NO: 2.

In another aspect, the present disclosure provides a method for performing a droplet digital PCR (ddPCR) assay on a sample of adeno-associated virus (AAV) genome prepared according to the process described above or herein, the method comprising: (a) performing the ddPCR assay on the sample of AAV genome loaded in a ddPCR plate with a ddPCR master mix; (b) analyzing data obtained from the ddPCR assay; and (c) determining a viral titer based on analysis of the data obtained from the ddPCR assay.

In some embodiments, the adeno-associated virus is of serotype AAV1, AAV5, or AAV8. In some embodiments, a threshold amplitude for analysis of the data is set at 6000.

In some embodiments, analysis of the data includes data with a copy number between 200 copies/μL and 10,000 copies/μL.

In some embodiments, the sample of AAV genome comprises an exogenous gene. In some cases, the exogenous gene is a therapeutic gene.

In some embodiments, determining the viral titer includes determining the viral titer of the exogenous gene in viral genomes/mL based on analysis of the data obtained from the ddPCR assay.

In some embodiments, the ddPCR assay produces a variability rate or a coefficient of variation of no more than 5%. In some cases, the ddPCR assay produces a variability rate or a coefficient of variation of less than 2%.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

Other embodiments will become apparent from a review of the ensuing detailed description.

### BRIEF DESCRIPTION OF THE DRAWINGS

FIG. 1 illustrates an AAV capsid with a single-stranded DNA genome comprising a therapeutic gene or gene of interest (GOI).

FIG. 2 illustrates an overview of an exemplary method for preparing a sample of viral particles and performing a ddPCR assay to quantify viral titer in accordance with an embodiment of the present disclosure.

FIG. 3 illustrates a plot for the relative standard deviation caused by stochastic effects in relation to the PCR copy number concentration for the ddPCR system (Deprez, et al., 2016, Biomol Detect Quantif 9:29-39), according to an embodiment of the present disclosure.

FIGS. 4A and 4B illustrate results of two ddPCR assays of viral titer in AAV particles using the sample preparation methods discussed herein. FIG. 4A is a graph of the data presented in tabular form in FIG. 4B, which also presents the % relative standard deviation.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “include,” “includes,” and “including,” are meant to be non-limiting and are understood to mean “comprise,” “comprises,” and “comprising,” respectively.

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Selected Abbreviations

AAV: Adeno-associated virus
rAAV: recombinant Adeno-associated virus particle or capsid
GOI: gene of interest
ddPCR: Droplet digital polymerase chain reaction
IPA: Isopropyl alcohol
EDTA: Ethylenediaminetetraacetic acid
dUTP: Deoxyuridine triphosphate
COA: Certificate of Analysis
RCF: Relative centrifugal force
NTC: No template control
bGH: Bovine growth hormone
RPM: Revolutions per minute
CV: Coefficient of Variation
ORF: Open reading frame
ITR: Inverted terminal repeat

### Definitions

“Adeno-associated virus” or “AAV” is a non-pathogenic parvovirus, with single-stranded DNA, a genome of approximately 4.7 kb, not enveloped and has icosahedric conformation. AAV was first discovered in 1965 as a contaminant of adenovirus preparations. AAV belongs to the Dependovirus genus and Parvoviridae family, requiring helper functions from either herpes virus or adenovirus for replication. In the absence of helper virus, AAV can set up latency by integrating into human chromosome 19 at the 19q13.4 location. The AAV genome consists of two open reading frames (ORF), one for each of two AAV genes, Rep and Cap. The AAV DNA ends have a 145-bp inverted terminal repeat (ITR), and the 125 terminal bases are palindromic, leading to a characteristic T-shaped hairpin structure.

The term “sample,” as used herein, refers to a mixture of viral particles (e.g., AAV particles) that comprises at least one viral capsid component encapsulating a single-stranded DNA genome that is subjected to manipulation in accordance with the methods of the invention, including, for example, dilution, treatment and ddPCR plating.

The term “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA), or modified or substituted sugar or phosphate groups.

A “recombinant viral particle” refers to a viral particle including one or more exogenous genes or heterologous sequences (e.g., a nucleic acid sequence not of viral origin) that may be flanked by at least one viral nucleotide sequence.

A “recombinant AAV particle” refers to an adeno-associated viral particle including one or more heterologous sequences (e.g., a nucleic acid sequence not of AAV origin) that may be flanked by at least one, for example two, AAV inverted terminal repeat sequences (ITRs). Such rAAV particles can be replicated and packaged when present in a host cell that has been infected with a suitable helper virus (or that is expressing suitable helper functions) and that is expressing AAV rep and cap gene products (i.e., AAV Rep and Cap proteins).

A “viral particle” refers to a viral particle composed of at least one viral capsid protein and an encapsulated viral genome.

“Heterologous” or “exogenous” means derived from a genotypically distinct entity from that of the rest of the entity to which it is compared or into which it is introduced or incorporated. For example, a nucleic acid introduced by genetic engineering techniques into a different cell type is a heterologous nucleic acid (and, when expressed, can encode a heterologous polypeptide). Similarly, a cellular sequence (e.g., a gene or portion thereof) that is incorporated into a viral particle is a heterologous or exogenous nucleotide sequence with respect to the viral particle.

The term “therapeutic gene” refers to a genetically modified gene that produces a therapeutic effect or the treatment of disease by repairing or reconstructing defective genetic material.

An “inverted terminal repeat” or “ITR” sequence is a relatively short sequence found at the termini of viral genomes which are in opposite orientation. An “AAV inverted terminal repeat (ITR)” sequence is an approximately 145-nucleotide sequence that is present at both termini of a single-stranded AAV genome.

The term “isolated,” as used herein, refers to a biological component (such as a nucleic acid, peptide, protein, lipid, viral particle or metabolite) that has been substantially separated, produced apart from, or purified away from other biological components in the cell of the organism in which the component naturally occurs or is transgenically expressed.

A “vector,” as used herein, refers to a recombinant plasmid or virus that comprises a nucleic acid to be delivered into a host cell, either in vitro or in vivo.

A “recombinant viral vector” refers to a recombinant polynucleotide vector including one or more heterologous sequences (i.e., nucleic acid sequence not of viral origin).

The term “corresponding” is a relative term indicating similarity in position, purpose or structure.

As used herein, “amplification” refers to the production of multiple copies of a segment of DNA or RNA. Amplification is usually induced by polymerase chain reaction.

As used herein, “PCR” refers to polymerase chain reaction which is a molecular biology technique used to amplify a single copy of a segment of DNA or RNA, generating thousands to millions of copies of a particular DNA or RNA sequence. PCR is commonly used to amplify the number of copies of a DNA or RNA segment for cloning or to be used in other analytical procedures.

As used herein, “primer-probe mix” refers to a grouping of a pair of oligonucleotide primers and an oligonucleotide probe that hybridize to a specific nucleotide sequence. The oligonucleotide set consists of: (a) a forward discriminatory primer that hybridizes to a first location of a nucleic acid sequence; (b) a reverse discriminatory primer that hybridizes to a second location of the nucleic acid sequence downstream of the first location and (c) a fluorescent probe labeled with a fluorophore and a quencher, which hybridizes to a location of the nucleic acid sequence between the primers. In other words, a primer-probe mix consists of a set of specific PCR primers capable of initiating synthesis of an amplicon specific to a nucleic acid sequence, and a fluorescent probe which hybridizes to the amplicon.

An “amplicon” refers to a nucleic acid fragment formed as a product of natural or artificial amplification events or techniques. For example, an amplicon can be produced by PCR, ligase chain reaction, or gene duplication.

A “probe” or “fluorescent probe” comprises an oligonucleotide sequence labeled with both a “fluorescent reporter dye”, or “fluorophore”, and a “quencher dye”, or “quencher.” A “fluorescent reporter dye” or “fluorophore” refers to a molecule that emits light of a certain wavelength after having first absorbed light of a specific, but shorter, wavelength, wherein the emission wavelength is always higher than the absorption wavelength. A “quencher dye” “quencher” refers to a molecule that accepts energy from a fluorophore in the form of light at a particular wavelength and dissipates this energy either in the form of heat (e.g., proximal quenching) or light of a higher wavelength than emitted from the fluorophore (e.g., FRET quenching). Quenchers generally have a quenching capacity throughout their absorption spectrum, but they perform best close to their absorption maximum. For example, Deep Dark Quencher II absorbs over a large range of the visible spectrum and, consequently, efficiently quenches most of the commonly used fluorophores, especially those emitting at higher wavelengths (like the Cy® dyes). Similarly, the Black Hole Quencher family covers a large range of wavelengths (over the entire visible spectrum and into the near-IR). In contrast, Deep Dark Quencher I and Eclipse® Dark Quencher effectively quench the lower wavelength dyes, such as FAM, but do not quench very effectively those dyes that emit at high wavelengths.

In embodiments, the fluorescent label on the oligonucleotide probe may be selected from the group of FAM (5- or 6-carboxyfluorescein), VIC, NED, Fluorescein, FITC, IRD-700/800, CY3, CY5, CY3.5, CY5.5, HEX, TET, TAMRA, JOE, ROX, BODIPY TMR, Oregon Green, Rhodamine Green, Rhodamine Red, Texas Red, Yakima Yellow, Alexa Fluor PET, Biosearch Blue™, Marina Blue®, Bothell Blue®, Alexa Fluor®, 350 FAM™, SYBR® Green 1, Fluorescein, EvaGreen™, Alexa Fluor® 488 JOE™, VIC™ HEX™ TET™, CAL Fluor® Gold 540, Yakima Yellow®, ROX™, CAL Fluor® Red 610, Cy3.5™, Texas Rede, Alexa Fluor® 0.568 Cy5™, Quasar™ 670, LightCycler Red640®, Alexa Fluor 633 Quasar™705, LightCycler Red705®, Alexa Fluor® 680, SYTO® 9, LC Green®, LC Green® Plus+, EvaGreen™ Fluorescent labels (i.e., dyes). The channel for detection and the excitation and detection wavelengths are provided in Table 1.

TABLE 1
Fluorophores and their channel for detection
and the excitation and detection wavelengths

| | Excitation | Detected |
|---|---|---|
| | source/ | Dyes |
| Channel | Detection filter | (Examples) |
| Blue | 365 ± 20 nm/ | Edans, Marina Blue ®, AMCA - X, |
| | 460 ± 15 nm | Atto390, Alexa Fluor ® 350 |
| Green | 470 ± 10 nm/ | FAM ™, Fluorescein, Cyan 500, Alexa |
| | 510 ± 5 nm | Fluor ® 488 |
| Yellow | 530 ± 5 nm/ | JOE ™, VIC ™, HEX ™, TET ™, |
| | 555 ± 5 nm | Yakima Yellow ®, Cal Fluor |
| | | Orange 560 |
| Orange | 585 ± 5 nm/ | ROX ™, Cy3.5 ®, Texas Red ®, Alexa |
| | 610 ± 5 nm | Fluor ® 568, CAL Fluor ™ Red 610 |
| Red | 625 ± 10 nm/ | Cy5 ®, Quasar 670 ™, LightCycler Red |
| | 660 ± 10 nm | 640 ®, Alexa Fluor ™ 633 |
| Crimson | 680 ± 5 nm/ | Quasar 705 ™, LC Red 705 ®, |
| | 712 long pass | LightCycler Red 670, Alexa Fluor ® 680 |

As used herein, “digital PCR” refers to an assay that provides an end-point measurement that provides the ability to quantify nucleic acids without the use of standard curves, as is used in real-time PCR. In a typical digital PCR experiment, the sample is randomly distributed into discrete partitions, such that some contain no nucleic acid template and others contain one or more template copies. The partitions are amplified to the terminal plateau phase of PCR (or end-point) and then read to determine the fraction of positive partitions. If the partitions are of uniform volume, the number of target DNA molecules present may be calculated from the fraction of positive end-point reactions using Poisson statistics, according to the following equation:

$$A = - 1 n ( 1 - p ) \qquad ( 1 )$$

wherein, λ is the average number of target DNA molecules per replicate reaction and p is the fraction of positive end-point reactions. Thus, together with the volume of each replicate PCR and the total number of replicates analyzed, an estimate of the absolute target DNA concentration is calculated. Digital PCR includes a variety of formats, including droplet digital PCR, BEAMing (beads, emulsion, amplification, and magnetic), and microfluidic chips.

“Droplet digital PCR” (ddPCR) refers to a digital PCR assay that measures absolute quantities by counting nucleic acid molecules encapsulated in discrete, volumetrically defined, water-in-oil droplet partitions that support PCR amplification (Hinson et al., 2011, Anal. Chem. 83:8604-8610; Pinheiro et al., 2012, Anal. Chem. 84:1003-1011). A single ddPCR reaction may be comprised of at least 10,000 (e.g., 20,000) partitioned droplets per well.

A “droplet” or “water-in-oil droplet” refers to an individual partition of the droplet digital PCR assay. A droplet supports PCR amplification of template molecule(s) using homogenous assay chemistries and workflows similar to those widely used for real-time PCR applications (Hinson et al., 2011, Anal. Chem. 83:8604-8610; Pinheiro et al., 2012, Anal. Chem. 84:1003-1011).

Droplet digital PCR may be performed using any platform that performs a digital PCR assay that measures absolute quantities by counting nucleic acid molecules encapsulated in discrete, volumetrically defined, water-in-oil droplet partitions that support PCR amplification. The strategy for droplet digital PCR may be summarized as follows: a sample is diluted and partitioned into thousands to millions of separate reaction chambers (water-in-oil droplets) so that each contains one or no copies of the nucleic acid molecule of interest. The number of “positive” droplets detected, which contain the target amplicon (i.e., nucleic acid molecule of interest), versus the number of “negative” droplets, which do not contain the target amplicon (i.e., nucleic acid molecule of interest), may be used to determine the number of copies of the nucleic acid molecule of interest that were in the original sample. Examples of droplet digital PCR systems include the QX200™ Droplet Digital PCR System by Bio-Rad, which partitions samples containing nucleic acid template into 20,000 nanoliter-sized droplets; and the RainDrop™ digital PCR system by RainDance, which partitions samples containing nucleic acid template into 1,000,000 to 10,000,000 picoliter-sized droplets. In embodiments, a ddPCR assay may provide an approach for accurate one-step quantification of nucleic acid. In embodiments, a ddPCR assay may also provide a multiplexed approach, wherein primers and sequence-specific oligonucleotide probes targeting more than one amplicon may be included. In a multiplexed ddPCR assay, instead of measuring individual loci, the assay relies on the average readout of all tested loci, measuring the concentration of nucleic acid with high precision and accuracy.

As used herein, “amplitude” refers to the maximum extent of a vibration or oscillation, measured from the position of equilibrium. In molecular biology, vortex mixers are one of the primary technologies for mixing laboratory samples in test tubes, well plates, or flasks. Vortex mixers use a simple mechanism to agitate or vibrate samples and encourage reactions or homogenization with high degrees of precision. In embodiments of the present invention, the amplitude of a vortex mixer may refer to the speed of agitation.

The term “revolutions per minute” or “RPM” refers to the number of turns in one minute. It is a unit of rotational speed or the frequency of rotation around a fixed axis. In other words, RPM is a measure of angular frequency.

The term “relative centrifugal force” or “RCF” refers to the amount of acceleration or centrifugal force exerted on a sample. RCF is also known as the g-force. Relative centrifugal force is dependent on the speed of rotation in RPM and the distance of the sample from the center of rotation. RCF is relative to the force of the earth's gravitational field and is expressed in multiples of the standard acceleration. RCF or g-force may be calculated using the following equation:

$$g ‐ Force = ( 1.118 \times 1 {0}^{- 5} ) \times r \times {S}^{2} \qquad ( 2 )$$

wherein, g-Force refers to the relative centrifugal force, r is the distance of the sample from the center of rotation, and S is the rotational speed in RPM. Doubling the speed of rotation increases the centrifugal force by a factor of four. The centrifugal force also increases with the distance from the center of rotation.

As used herein, “coefficient of variation” is the ratio of the standard deviation to the mean and shows the extent of variability of data in a sample in relation to the mean of the population. The higher the coefficient of variation, the greater the dispersion. In embodiments of the present invention involving a ddPCR assay, the coefficient of variation (CV %) of a sample is calculated to reflect reproducibility from run-to-run. The CV % may be calculated using the formula: Coefficient of Variation=(Standard Deviation/Mean)*100. Multiplying the coefficient by 100 produces a percentage, as opposed to a decimal.

The term “copy number” refers to the number of copies of a particular gene present in the genome of an organism. Genetic variants, including insertions, deletions, and duplications of segments of DNA, are collectively referred to as copy number variants. Copy number variation is defined as the presence of variable numbers of copies of a particular DNA segment relative to a reference genome. Digital PCR permits very high-resolution determination of copy number variation.

### General Description

The present disclosure provides methods for preparing a sample of viral genome (e.g., AAV genome) and performing a droplet digital PCR assay on the prepared sample of viral genome. The current methods enable quantification of viral genome by ddPCR assay. The methods of viral genome quantification in accordance with the present disclosure includes sample dilution, DNase I/Proteinase K treatment, serial dilution and treatment with ddPCR master mix, and analysis of data obtained from the ddPCR assay. In embodiments, the analysis quantifies viral genomes present inside the capsids of recombinant adeno-associated viruses (rAAV) samples using primer-probes specific to recombinant viral payload. The droplet digital PCR assay in accordance with the present disclosure produces a coefficient of variation of no more than 5% (e.g., less than 2%).

### Methods for Preparing a Sample of Viral Genome for Droplet Digital PCR Assay

Aspects of the disclosure are directed to methods of preparing a sample of viral genome from a sample of viral particles (e.g., recombinant AAV particles) for a droplet digital PCR (ddPCR) assay.

In some cases, the method comprises: (a) diluting the sample of viral genome with a dilution buffer and gently mixing the diluted sample of viral genome with a relative centrifugal force ranging from about 10 to about 150 (e.g., 20-100) for a period of from 10 to 45 seconds (e.g., 15-20 seconds); (b) treating the diluted sample of viral genome with DNase I by gently mixing with a DNase I reaction mix at a relative centrifugal force of from about 10 to about 150 (e.g., 20-100) for a period of from 10 to 45 seconds (e.g., 15-20 seconds); (c) treating the DNase I-treated sample of viral genome with Proteinase K by mixing with a Proteinase K reaction mix at a relative centrifugal force of from about 50 to about 300 (e.g., 100-200) for a period of from 10 to 45 seconds (e.g., 15-20 seconds); (d) serially diluting the Proteinase K-treated sample of viral genome with the dilution buffer and mixing the serially diluted sample of viral genome with a relative centrifugal force of from about 50 to about 300 (e.g. 100-200) for a period of from 10 to 45 seconds (e.g., 15-20 seconds); (e) preparing a master mix comprising a primer-probe mix for the ddPCR assay and mixing the prepared master mix at a relative centrifugal force of from about 50 to about 300 (e.g., 100-200) for a period of from 10 to 45 seconds (e.g., 15-20 seconds); and (f) loading the serially diluted sample of viral genome with the master mix comprising the primer-probe mix on a ddPCR plate for the ddPCR assay with a relative centrifugal force of from about 50 to about 300 (e.g. 100-200) for a period of from 10 to 45 seconds (e.g., 15-20 seconds).

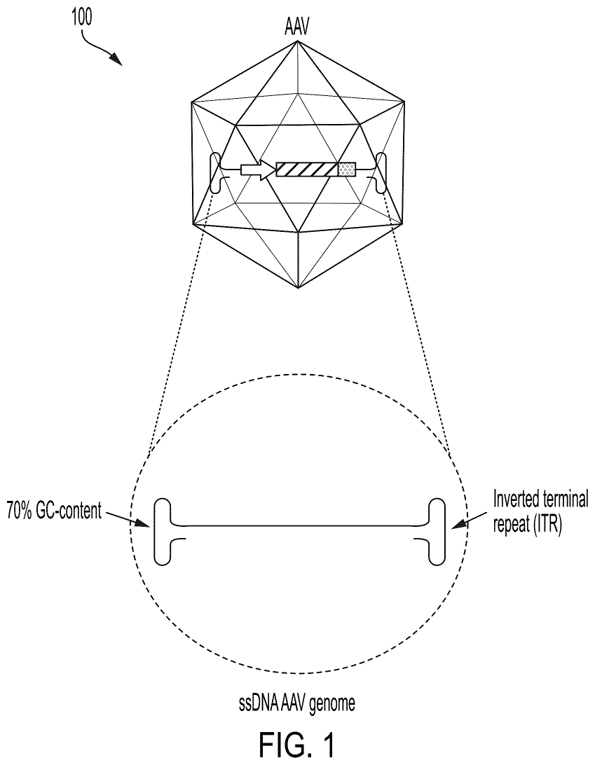

An adeno-associated viral particle 100 along with its genome (e.g., single-stranded DNA genome) is illustrated in FIG. 1. In the example shown in FIG. 1, the genome of AAV is highly symmetrical with palindromic elements. In addition, the genome of AAV comprises approximately 70% GC-content with inverted terminal repeats. In some examples, the genome of a recombinant AAV may comprise a therapeutic gene or gene of interest (GOI) for the purposes of gene therapy, for example.

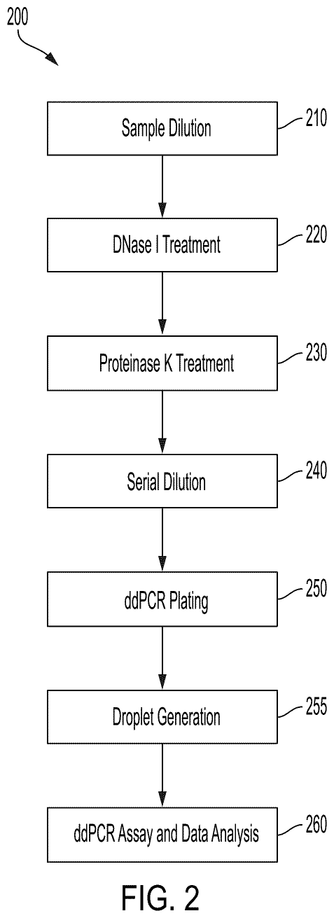

The schematic illustrated in FIG. 2 provides an overview of an exemplary method 200 for preparing a sample of viral genome and assaying viral titer by droplet digital PCR assay. In the example method 200, the sample of viral genome is first diluted with a dilution buffer at step 210. In one example, the dilution ratio may be 1:9 (sample:dilution buffer). In some cases, the dilution ratio may be 1:8 or 1:10. The dilution buffer may comprise a non-ionic surfactant (e.g., 0.05% Pluronic F-68), Tris HCl (pH 8.0), and a sheared salmon sperm DNA. In embodiments, the diluted sample of viral genome is gently mixed using a vortex mixer. The RCF values for gentle mixing may range from about 10 to about 150 (e.g., 20-100). The diluted sample of viral genome is then subjected to DNase I treatment at step 220. The diluted sample of viral genome is gently mixed with a freshly prepared DNase I reaction mix. The RCF values for gentle mixing using a vortex mixer may range from about 10 to about 150 (e.g., 20-100). The DNase I reaction is performed at a temperature ranging from 35° C. to 40° C. (e.g., at 37° C.) for about 25-35 minutes (e.g., 30 minutes).

At step 230, the DNase I-treated sample of viral genome is then subjected to Proteinase K treatment. In examples, the DNase I-treated sample of viral genome is mixed with a freshly prepared Proteinase K reaction mix. The RCF values for mixing using a vortex mixer may range from about 50 to about 300 (e.g., 100-200). The Proteinase K reaction is performed at a temperature ranging from 50° C. to 60° C. (e.g., at 55° C.) for about 25-35 minutes (e.g., 30 minutes), followed by heating at a temperature ranging from 90° C. to 100° C. (e.g., at 95° C.) for about 10-20 minutes (e.g., 15 minutes). Subsequently, at step 240, the DNase I/Proteinase K-treated sample of viral genome is serially diluted with the dilution buffer. In examples, the dilution factor may be 100K-1M (final dilution factor), for example. The serially diluted sample of viral genome is mixed with a vortex mixer at an RCF of from about 50 to about 300 (e.g., 100-200). A master mix comprising a primer-probe mix is prepared for ddPCR assay. The prepared master mix is mixed using a vortex mixer at an RCF of from about 50 to about 300 (e.g., 100-200). At step 250, the serially diluted sample of viral genome is mixed with the ddPCR master mix and loaded onto a ddPCR plate. The RCF values for mixing using a vortex mixer may range from about 50 to about 300 (e.g., 100-200). The mixing is conducted before loading on the ddPCR plate as well as after sealing of the plate. Subsequently, at step 255, droplet generation is performed in the samples loaded in the ddPCR plate. In some examples, the droplets may be generated using an eight-channel droplet generator cartridge present within a droplet generator. The replicates may be transferred to separate wells within a single column of the ddPCR plate for thermal cycling and droplet reading. Finally, the ddPCR is performed at step 260, and details regarding the ddPCR assay and data analysis are presented in the next section below.

In various embodiments of the methods discussed herein, the buffers such as the dilution buffer, 1% Pluronic F-68, and Proteinase K buffer are filtered through a 0.22 μm (e.g., from 0.2 to 0.25 μm) syringe filter (PVDF) to remove any particulates that may affect the ddPCR assay. Furthermore, in embodiments, the mixing or gentle mixing of sample is conducted in a Lo-Bind tube or Lo-Bind 96-well plate having an inner wall with reduced nucleic acid adsorption. Additionally, sample mixing is performed using vortex mixer, instead of pipetting. This eliminates sample adsorption on sampling tips through pipetting and improves mixing efficiency.

In various embodiments of the methods discussed herein, gentle mixing of a sample using a Vortex-Genie 2 mixer refers to an RPM value ranging from about 1367 to about 1567 (e.g., 1467 RPM). Additionally, in embodiments, mixing of a sample using a Vortex-Genie 2 mixer refers to an RPM value ranging from about 2234 to about 2434 (e.g., 2334 RPM). These mixing speeds equate to relative centrifugal force (RCF) values calculated as discussed herein. For example, RCF values may be calculated as a function of the RPM and the distance of the sample from the center of rotation, according to Equation 2 described above. In the illustrated example, the size of the plate or platform of a Vortex-Genie 2 mixer is about 3 inches in diameter. Thus, if the sample is placed anywhere between the center and the edge of the plate, a corresponding RCF value may be calculated using Equation 2. For example, if the sample is placed at a distance of about 1 inch from the center of rotation of the 3-inch platform and the sample is spinning at about 1467 RPM, the corresponding RCF value calculated using Equation 2 would be 60 for gentle mixing of the sample. As another example, if the sample is placed at a distance of about 1 inch from the center of rotation of the 3-inch platform and the sample is spinning at about 2334 RPM, the corresponding RCF value calculated using Equation 2 would be 153 for mixing of the sample. A broader range for relative centrifugal force values for each of mixing and gentle mixing of a sample is described below in accordance with various embodiments of the present disclosure.

In particular examples, a value for relative centrifugal force (RCF) for gentle mixing of a sample using a vortex mixer may be within a range of from about 10 to about 200 (e.g., from about 60-190), or about 10 to about 150, or from about 20 to about 100 (e.g., about 25 to about 75), about 50 to about 150, or about 40 to about 60, particularly about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, or about 200. In specific embodiments, these RCF values for gentle mixing of a sample may be converted back to RPM values using Equation 2 for a specific vortex mixer based on a location of the sample from center of rotation. For example, an RCF value of 60 for gentle mixing using a Vortex-Genie 2 mixer may be equivalent to an RPM value of 1467, when the sample is placed on a platform of the mixer at a distance of about 1 inch from the center of rotation.

In particular examples, a value for relative centrifugal force (RCF) for mixing of a sample using a vortex mixer may be within a range of from about 50 to about 500, about 50 to about 300 (e.g., from about 100-200 or 110-160), or about 110 to about 140, particularly about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500. In specific embodiments, these RCF values for mixing of a sample may be converted back to RPM values using Equation 2 for a specific vortex mixer based on a location of the sample from center of rotation. For example, an RCF value of 153 for mixing of a sample using a Vortex-Genie 2 mixer may be equivalent to an RPM value of 2334, when the sample is placed on a platform of the mixer at a distance of about 1 inch from the center of rotation.

In particular examples, mixing (whether gentle mixing or mixing as discussed herein) may be performed for a specified period of time. In some cases, the period of time may range from about 5 seconds to about 120 seconds. In some case, the period of time may range from about 10 second to about 60 seconds, or from about 10 seconds to about 45 seconds. In various embodiments, the period of time may be, or be about, 5 seconds, 10 seconds, 15 seconds, 20 seconds, 25 seconds, 30 seconds, 35 seconds, 40 seconds, 45 seconds, 50 seconds, 55 seconds, 60 seconds, 70 seconds, 80 seconds, 90 seconds, 100 seconds, 110 seconds, or 120 seconds. In some cases, the period of time is about 15 seconds.

### Methods for Performing Droplet Digital PCR Assay on a Sample of Viral Genome

Aspects of the disclosure are directed to methods of performing a droplet digital PCR assay on a sample of adeno-associated virus (AAV) genome (e.g., the sample of viral genome from rAAV particles) prepared according to the process described above.

In some cases, the method comprises: (a) performing the ddPCR assay on the sample of AAV genome loaded in a ddPCR plate with a ddPCR master mix; (b) analyzing data obtained from the ddPCR assay; and (c) determining a viral titer based on analysis of the data obtained from the ddPCR assay.

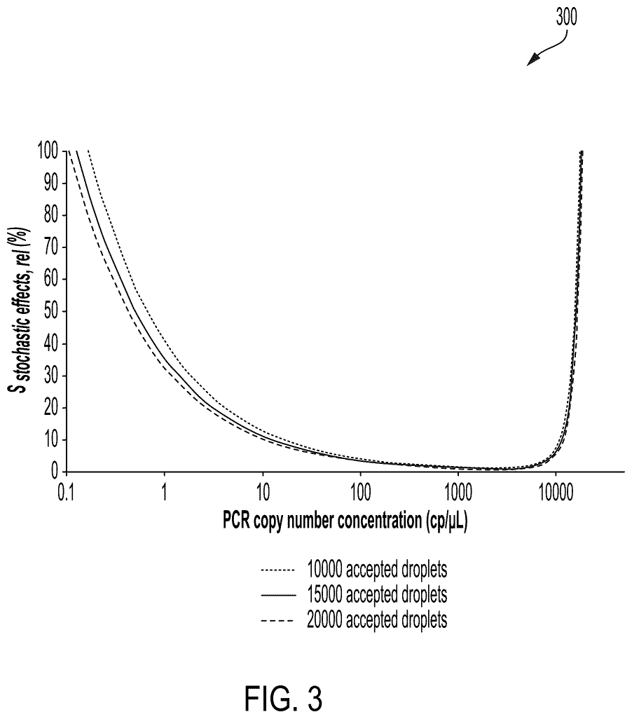

In embodiments of the methods discussed herein, a droplet digital PCR assay is carried out on the sample of AAV genome loaded in the ddPCR plate with the ddPCR master mix, and the data from the ddPCR assay is collected. For analysis of the data, a threshold amplitude may be set at −5000-6000 amplitudes on a QX-200 device. Although the exact threshold amplitude may be device/primer-probe mix specific, the concept of using higher threshold may apply in general to ddPCR assays, which can help reduce assay variability. Analysis of the data includes data with a copy number between 200 copies/μL and 8000 copies/μLto minimize stochastic effect on highly diluted sample. A plot is provided in FIG. 3 illustrating the relative standard deviation caused by stochastic effects in relation to the PCR copy number concentration for the ddPCR system.

In embodiments, a viral titer may be determined based on analysis of the data obtained from the ddPCR assay. In some examples, wherein the sample of AAV genome comprises an exogenous gene (e.g., a therapeutic gene), determining the viral titer may include determining the viral titer of the exogenous gene in viral genomes/mL based on analysis of the data obtained from the ddPCR assay.

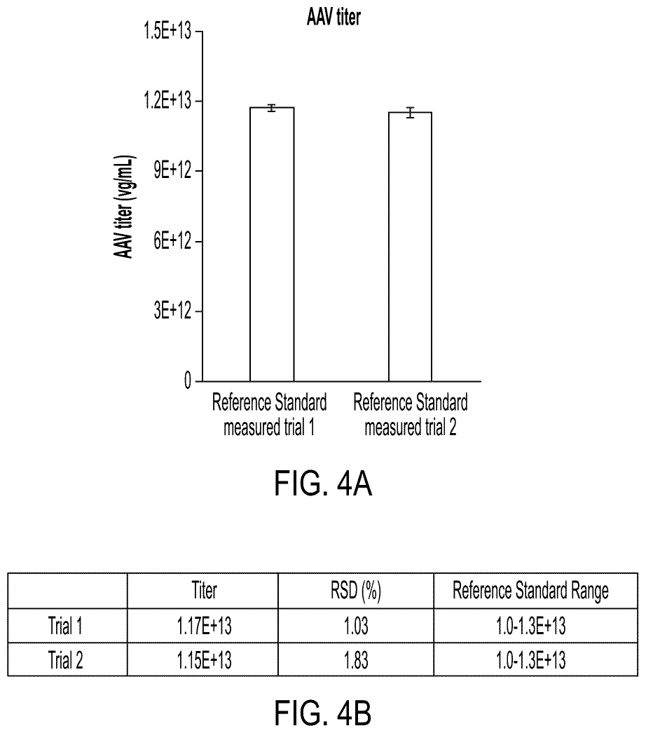

In embodiments of the methods disclosed herein, the droplet digital PCR assay produces a coefficient of variation of no more than 5%. In specific examples, the ddPCR assay in accordance with the present disclosure produces a coefficient of variation of less than 2% (e.g., 1.83%). FIG. 4A illustrates a plot for an AAV titer obtained according to the methods of the present disclosure (e.g., the method shown in FIG. 2). FIG. 4B provides a table indicating improvement in assay variability with a coefficient of variation of approximately 2%. In particular examples, the ddPCR assay in accordance with the present disclosure may produce a coefficient of variation of about 0.5%, about 1%, about 1.5%, about 2%, about 2.5%, about 3%, about 3.5%, about 4%, about 4.5%, or about 5%. This is in contrast to the high variability rate of approximately 14% of a conventional ddPCR assay in which sample preparation includes mixing by pipetting (e.g., at least 25 times) rather than mixing as discussed herein. Thus, the present method improves the performance of the ddPCR assay by reducing the variability rate. Additionally, the present method enables identification of changes in the viral titer much more specifically than other methods.

### Viral Particles

In certain aspects, the viral particle is an AAV particle and the methods disclosed can be used to quantify viral genomes present inside the capsids of a sample of AAV particles. The AAV particles may be recombinant AAV (rAAV) particles. The rAAV particle includes a heterologous transgene or heterologous nucleic acid molecule.

In certain aspects, the AAV particles include an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAV11 capsid, an AAV 12 capsid, or a variant thereof. In certain aspects, the AAV particles are of serotype AAV1, AAV2, AAV3, AAV4, AAV5, AAV6, AAV7, AAV8, AAV9, AAV-DJ, AAV-DJ/8, AAV-Rh10, AAV-retro, AAV-PHP.B, AAV8-PHP.eB, or AAV-PHP.S. In some embodiments, the AAV particles are of serotype AAV1 or AAV8.

While AAV was the model viral particle for this disclosure, it is contemplated that the disclosed methods can be applied to characterize a variety of viruses, for example, the viral families, subfamilies, and genera. The methods of the present disclosure may find use, for example, in quantifying viral genomes to determine a viral titer of a gene of interest present in the viral capsids of a composition of viral particles during production, purification or storage of such compositions.

In exemplary embodiments, the viral particle belongs to a viral family selected from the group consisting of Adenoviridae, Parvoviridae, Retroviridae, Baculoviridae, and Herpesviridae.

In certain aspects, the viral particle belongs to a viral genus selected from the group consisting of Atadenovirus, Aviadenovirus, Ichtadenovirus, Mastadenovirus, Siadenovirus, Ambidensovirus, Brevidensovirus, Hepandensovirus, Iteradensovirus, Penstyldensovirus, Amdoparvovirus, Aveparvovirus, Bocaparvovirus, Copiparvovirus, Dependoparvovirus, Erythroparvovirus, Protoparvovirus, Tetraparvovirus, Alpharetrovirus, Betaretrovirus, Deltaretrovirus, Epsilonretrovirus, Gammaretrovirus, Lentivirus, Spumavirus, Alphabaculovirus, Betabaculovirus, Deltabaculovirus, Gammabaculovirus, Iltovirus, Mardivirus, Simplexvirus, Varicellovirus, Cytomegalovirus, Muromegalovirus, Proboscivirus, Roseolovirus, Lymphocryptovirus, Macavirus, Percavirus, and Rhadinovirus.

In certain aspects, the Retroviridae is Moloney murine sarcoma virus (MOMSV), Harvey murine sarcoma virus (HaMuSV), murine mammary tumor virus (MuMTV), gibbon ape leukemia virus (GaLV), feline leukemia virus (FLV), Spumavirus, Friend virus, Murine Stem Cell Virus (MSCV) Rous Sarcoma Virus (RSV), human T cell leukemia viruses, Human Immunodeficiency Viruse (HIV), feline immunodeficiency virus (FIV), equine immunodeficiency virus (EIV), visna-maedi virus; caprine arthritis-encephalitis virus; equine infectious anemia virus; feline immunodeficiency virus (FIV); bovine immune deficiency virus (BIV); or simian immunodeficiency virus (SIV).

In some aspects, the viral particle (e.g., AAV particle) contains a heterologous nucleic acid molecule or exogenous gene (e.g., a therapeutic gene or gene of interest). In some aspects, the heterologous nucleic acid molecule is operably linked to a promoter. Exemplary promoters include, but are not limited to, the cytomegalovirus (CMV) immediate early promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit.beta.-globin promoter and the elongation factor 1-alpha promoter (EF1-alpha) promoter. In some aspects, the promoter comprises a human.beta.-glucuronidase promoter or a cytomegalovirus enhancer linked to a chicken.beta.-actin (CBA) promoter. The promoter can be a constitutive, inducible or repressible promoter. In some aspects, the invention provides a recombinant vector comprising a nucleic acid encoding a heterologous transgene of the present disclosure operably linked to a CBA promoter. In some cases, the native promoter, or fragment thereof, for the transgene will be used. The native promoter can be used when it is desired that expression of the transgene should mimic the native expression. The native promoter may be used when expression of the transgene must be regulated temporally or developmentally, or in a tissue-specific manner, or in response to specific transcriptional stimuli. In a further aspect, other native expression control elements, such as enhancer elements, polyadenylation sites or Kozak consensus sequences may also be used to mimic the native expression.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: AAV Genome Quantification by Droplet Digital PCR Assay

AAV samples of different serotypes were prepared in-house. The total nucleic acid (i.e., viral genome) was extracted from AAV cultured in a human host. The methods of viral genome quantification, according to the present disclosure, includes sample dilution, DNase I/Proteinase K treatment, and analysis of the samples by droplet digital PCR assay using a QX-200 instrument (Bio-Rad, Hercules, CA). This analysis quantifies viral genomes present inside the capsids of recombinant adeno-associated viruses (rAAV) samples using primer-probes specific to recombinant viral payload. Additionally, the methods of the present disclosure may be utilized to characterize in-process samples, drug substance, and drug product as well as stability studies.

### Chemicals and Reagents

Unless otherwise stated, all chemicals and reagents were acquired from MilliporeSigma (Burlington, MA, USA). AAV samples and their nucleic acid extract were provided in-house (Regeneron Pharmaceuticals Inc., Tarrytown, NY, USA). QX-200 Droplet Generator, QX-200 Droplet Reader, PX1-PCR Plate Sealer, PCR Plate Heat Seal, foil, C1000 Touch Thermocycler, 96 well DNA Lo-Bind PCR Plate, ddPCR Supermix for Probes, QX-ONE Droplet Generation Oil for Probes, and ddPCR™ Droplet Reader Oil were acquired from Bio-Rad (Hercules, CA). Vortex-Genie 2 Mixer was acquired from Scientific Industries (Bohemia, NY). Minicentrifuge, Microcentrifuge, 1.5 ml/5 ml DNA/RNA Lo-Bind Tubes, Isopropanol, and 6% Peroxide were acquired from VWR (Atlanta, GA, USA). Pluronic F-68 was acquired from Thermo Fisher Scientific (Waltham, MA, USA). Proteinase K Solution (20 mg/mL) and EDTA were purchased from Invitrogen (Waltham, MA, USA). Nuclease-free Water, DNase I, DNase I Buffer (10×), SDS, Sheared Salmon Sperm DNA (10 mg/mL), and Tris HCl (pH 8.0) were purchased from Ambion (Austin, TX).

TABLE 2
Specific Reagents

| Name | Stock Concentration | Source |
|---|---|---|
| AAV in-house reference standard | Refer COA | Regeneron |
| Forward sequence detection primer | 9 μM | Bio-Rad |
| working stock solution (SEQ ID NO: 1) | | (Hercules, |
| 5′-GCCAGCCATCTGTTGT-3′ | | CA) |
| Reverse sequence detection primer | 9 μM | Bio-Rad |
| working stock solution (SEQ ID NO: 2) | | (Hercules, |
| 5′-GGAGTGGCACCTTCCA-3′ | | CA) |
| TaqMan fluorescent probe working stock | 2.5 μM | Bio-Rad |
| solution (SEQ ID NO: 3) | | (Hercules, |
| 5′-6FAM-TCCCCCTTGCCTTCCTTGACC- | | CA) |
| BHQ-3′ | | |

Note:
20x ddPCR Primer-Probe mixture containing the forward and reverse primers can be used directly

### Buffer Preparation

1% Pluronic F-68 Preparation: 1% Pluronic solution was prepared in nuclease-free water according to Table 3 below. The solution was mixed by gently inverting the tube at least 5 times. The solution was filtered with a 0.22 μm syringe filter (PVDF) and aliquoted into 1 mL/1.5 mL DNA Lo-Bind tubes. The solution may be stored at −20° C. after use. There are no limits on freeze-thaw cycle.

TABLE 3
1% Pluronic F-68 Preparation

| | Volume (mL) |
|---|---|
| Pluronic F-68, 10% solution | 1 |
| Nuclease-free sterile water | 9 |
| Total | 10 |

Dilution Buffer Preparation: Sample dilution buffer solution was prepared in nuclease-free water according to Table 4 below. The solution was mixed by gently inverting the tube at least 5 times. The solution was filtered with a 0.22 μm syringe filter (PVDF) and aliquoted into 1 mL/1.5 mL DNA Lo-Bind tubes. The solution may be stored at −20° C. after use. There are no limits on freeze-thaw cycle.

TABLE 4
Dilution Buffer Preparation

| | | Final |
|---|---|---|
| Reagent | Volume (μL) | Concentration |
| 1M Tris HCl, pH 8.0 | 800 | 20 mM |
| 10% Pluronic F-68 | 200 | 0.05% |
| Sheared Salmon Sperm | 8 | 0.002 μg/μL |
| DNA (10 μg/uL) | | |
| Nuclease-free water | 38,992 | N/A |
| Total | 40,000 | |

10×Proteinase K Buffer Preparation: Proteinase K buffer (10×) solution was prepared in nuclease-free water according to Table 5 below. The solution was mixed by gently inverting the tube at least 5 times. The solution was filtered with a 0.22 μm syringe filter (PVDF) and aliquoted into 1 mL/1.5 mL DNA Lo-Bind tubes. The solution may be stored at −20° C. after use. There are no limits on freeze-thaw cycle.

TABLE 5
10X Proteinase K Buffer Preparation

| Reagent | Volume (mL) |
|---|---|
| SDS 10% Solution | 1 |
| 0.5M EDTA | 2 |
| 1M Tris pH 8.0 | 1 |
| Nuclease Free Water | 6 |
| Total | 10 |

### Initial Sample Dilution

Three replicates of each sample and in-house reference standard were tested for each instance of the assay. The samples and standards were equilibrated to room temperature. Each sample was mixed by inverting tube for 10 times. The tubes were spun down briefly thereafter to collect liquid at the bottom using microfuge. 90 μL of Dilution Buffer, as prepared above according to Table 4, were added to 1.5 mL DNA Lo-Bind tubes. Subsequently, 10 μL of sample/in-house reference standard were added to corresponding tubes containing dilution buffer and gently mixed with Vortex mixer for 15 seconds (amplitude: 4). The RCF values for gentle mixing may range from about 40 to about 60 (e.g., 45-55). The diluted samples/in-house reference standard were then spun down quickly using a mini centrifuge.

### DNase I Reaction Setup

DNase I reaction mix was freshly prepared in nuclease-free water according to Table 6 below. 30 μL of DNase I reaction mix was added to corresponding sample wells of a new Lo-Bind 96-well plate. Subsequently, 20 μL of diluted samples/in-house reference standard were transferred to 96-well plate containing DNase I reaction mix. The 96-well plate was sealed with a heat seal foil using PX1 Sealer at 180° C. for 3 seconds. The reaction mix within the sealed 96-well plate was then gently mixed by using Vortex mixer for 15 seconds (amplitude: 4). The RCF values for gentle mixing may range from about 40 to about 60 (e.g., 45-55). The 96-well plate was spun down at 1000 RCF for 1 minute. The 96-well plate was finally incubated on thermal cycler at 37° C. for 30 minutes followed by an infinite hold at 4° C. after. Before use, the 96-well plate may be equilibrated to room temperature and briefly spun down at 1000 RCF for 1 minute.

TABLE 6
DNase I Reaction Mix

| | Volume for one |
|---|---|
| Reagent | Reaction (μL) |
| Nuclease-free water | 17 |
| 10X DNase I Reaction Buffer | 5 |
| 1% Pluronic | 5 |
| DNAse I | 3 |
| Total | 30 |

### Proteinase K Reaction

Proteinase K reaction mix was freshly prepared in nuclease-free water according to Table 7 below. 50 μL Proteinase K reaction mix was transferred to corresponding sample wells of the DNase I digested 96-well plate. The 96-well plate was then sealed with a heat seal foil using PX1 Sealer at 180° C. for 3 seconds. The reaction mix within the sealed 96-well plate was mixed by Vortex mixer for 15 seconds (amplitude: 7). The RCF values for mixing, in this case, may range from about 110 to about 140 (e.g., 120-130). The 96-well plate was spun down at 1000 RCF for 1 minute. The 96-well plate was finally incubated at 55° C. for 30 minutes, followed by 15 minutes at 95° C. and then an infinite hold at 4° C. after. Before use, the treated 96-well plate may be equilibrated to room temperature and briefly spun down at 1000 RCF for 1 minute. (If ddPCR is not set up immediately, the treated sample plate may be stored at 4° C. for up to 24 hours.)

TABLE 7
Proteinase K Reaction Mix

| | Volume for one |
|---|---|
| Reagent | reaction (μL) |
| Nuclease-free water | 32 |
| 10X Proteinase K Buffer | 10 |
| 1% Pluronic | 5 |
| Proteinase K | 3 |
| Total | 50 |

### Serial Dilution Setup

Serial dilutions were performed in DNA Lo-Bind tubes. 90 μL of dilution buffer were added to fresh 1.5 mL DNA Lo-Bind tubes. Subsequently, 10 μL of DNase I/Proteinase K treated samples from the 96-well plate were transferred to corresponding tubes containing dilution buffer and were mixed. 10-fold dilutions of the treated samples were performed as required for each sample replicate (e.g., 10, 100, 1000, 10,000, 100,000, etc.). A dilution factor screening was performed when handling the samples with unknown titer to identify suitable dilution factor for ddPCR. When making serial dilutions, samples were mixed using Vortex mixer for 10-15 seconds (amplitude: 7) and quickly spun down with mini centrifuge. The RCF values for mixing may range from about 110 to about 140 (e.g., 120-130). Two dilutions were included for each ddPCR analysis. Since the titer of the reference standard will be known, a single dilution may be used.

### Droplet Digital PCR Master Mix Preparation

Each of the primer-probe mix and ddPCR Supermix was equilibrated to room temperature and vortexed for at least 15 seconds. Droplet digital PCR master mix was freshly prepared in nuclease-free water according to Table 8 below. The ddPCR master mix was vortexed using Vortex mixer for 15 seconds (amplitude: 7) and quickly spun down with mini centrifuge. The RCF values for mixing may range from about 110 to about 140 (e.g., 120-130).

TABLE 8
ddPCR Master Mix Preparation

| | Volume per |
|---|---|
| Reagent | well (μL) |
| 2x ddPCR Supermix for Probes | 11 |
| 20x Primer-Probe Mix | 1.1 |
| Nuclease free water | 5.5 |
| Sample | 4.4 |
| Total Volume | 22 |

Preparation of plate for ddPCR

17.6 μL of the ddPCR master mix (prepared above) was pipetted to each well of a brand new Lo-bind 96-well plate. Subsequently, 4.4 μL of appropriate dilutions for the treated samples/reference standard were transferred to corresponding wells of the 96-well plate containing the ddPCR master mix. Before loading the treated AAV genome samples/reference standard to the plate, they were mixed again with Vortex mixer for 10 seconds (amplitude: 7) and quickly spun down with mini centrifuge. The RCF values for mixing may range from about 110 to about 140 (e.g., 120-130).

A non-template control (NTC) was prepared by adding 4.4 μL of dilution buffer to corresponding wells of the 96-well plate containing the ddPCR master mix. The 96-well plate was then sealed with a heat seal foil using PX1 Sealer at 180° C. for 3 seconds. After sealing the plate, the samples within the sealed 96-well plate were mixed using Vortex mixer for 15 seconds (amplitude: 7). The RCF values for mixing may range from about 110 to about 140 (e.g., 120-130). The 96-well plate was then centrifuged or spun down at 1000 RCF for 1 minute.

### Loading and Configuring Plate in QX-200 Droplet Generator

The heat sealed 96-well plate was loaded into the QX200 Droplet Generator and the plate was configured by selecting the appropriate wells to be run. Once the rest of the consumables were loaded, the QX200 droplet generator was run. After droplet generation was completed, the plate was sealed with a heat seal foil using PX1 Sealer at 180° C. for 3 seconds. It was ensured that the plate was sealed within 30 minutes of the completion of droplet generation.

### PCR Thermal Cycling

The heat sealed 96-well plate was then loaded onto a C1000 deep well thermal cycler after droplet generation. The thermal cycling conditions were selected according to Table 9 below. The selection of thermal cycling conditions is specific to primer-probe mixture used. After the completion of PCR thermal cycling, the plate may be held on thermal cycling at 4° C. for up to 24 hours or may be removed to move forward with the next step.

TABLE 9
PCR Thermal Cycling Conditions

| Step | Temperature | Time | Cycles | Ramp Rate |
|---|---|---|---|---|
| 1 | 25° C. | 3 minutes | 1 | 2.5° C./s |
| 2 | 95° C. | 10 minutes | 1 | |
| 3 | 95° C. | 30 seconds | 40 | |
| | 60° C. | 1 minute | | |
| 4 | 98° C. | 10 minutes | 1 | |
| 5 | 4° C. | Infinite hold | 1 | |

### Loading and Configuring Plate in QX-200 Droplet Reader

Subsequent to PCR thermal cycling, the 96-well plate may be transferred and loaded onto a QX-200 Droplet Reader. The plate was configured by selecting the appropriate wells to be run in the QX Manager Software. Appropriate sample descriptions for selected wells were added. An example sample description for a selected well is shown in Table 10 below. Once the plate layout was configured, the QX-200 droplet reader was run.

TABLE 10
Example sample description for a selected well

| Experiment Type | Direct Quantification (DQ) |
|---|---|
| Sample Description 1 | Sample 1 |
| Sample Description 2 | Replicate 1 |
| Sample Description 3 | Dilution - 100 |
| Sample Description 4 | N/A |
| Sample Type | Unknown |
| Supermix | ddPCR Supermix for Probes (No dUTP) |
| Assay Type | Single Target per Channel |
| Target 1 | bGH-PolyA, FAM |

### Data Analysis and Calculations

Subsequent to the completion of the run of QX-200 droplet reader, the data collected was analyzed in the Analysis module of QX Manager Software. A threshold amplitude was manually set up to separate positive and negative droplets. For bGH-polyA probes, the threshold may be set at amplitude 4000. In the illustrated example, however, the threshold amplitude was set at 6000 due to high background signal for the primer-probe mix. Finally, the raw data was exported for further analysis and calculations.

As a system suitability criterion, all wells having more than 10,000 droplets were considered acceptable and were included in final analysis. Copies/μL in NTC wells should be less than 5. For samples and reference standards, all dilutions with copy number between 200-8000 copies/μL were included. To calculate viral titer in viral genomes/mL (vg/mL), concentration in copies/μL may be multiplied with the dilution factors, as shown below:

TABLE 11
Dilution Factors

| Step | Dilution Factor |
|---|---|
| Initial dilution | 10 |
| DNase I | 2.5 |
| Proteinase K | 2 |
| ddPCR master mix | 5 |
| μL to mL | 1000 |
| Sample dilution | Example, For the selected well |
| | (of Table 10) use 100 |

$$AAV Titer vg / mL = Concentration ( copies / μL ) \times 10 \times 2.5 \times 2 \times 5 \times 1000 \times sample dilution factor \qquad ( 3 )$$

The mean concentration for each unknown sample was calculated using Equation 3 above. Finally, the mean, standard deviation, and % CV for samples and reference standards were reported.

### Results and Discussion

According to the methods disclosed herein, the droplet digital PCR assay produces a coefficient of variation of no more than 5%. In specific examples, the ddPCR assay in accordance with the present disclosure produces a coefficient of variation of less than 2% (e.g., 1.83%). FIG. 4A illustrates a plot for an AAV titer obtained according to the methods of the present disclosure (e.g., the method shown in FIG. 2). FIG. 4B provides a table indicating improvement in assay variability with a coefficient of variation of approximately 2%. This is in contrast to the high variability rate of approximately 14% of a conventional ddPCR assay.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A method of preparing a sample of viral genome from a sample of viral particles for a droplet digital PCR (ddPCR) assay, comprising:
(a) diluting the sample of viral genome with a dilution buffer and gently mixing the diluted sample of viral genome with a relative centrifugal force ranging from about 10 to about 150;
(b) treating the diluted sample of viral genome with DNase I by gently mixing with a DNase I reaction mix at a relative centrifugal force of from about 10 to about 150;
(c) treating the DNase I-treated sample of viral genome with Proteinase K by mixing with a Proteinase K reaction mix at a relative centrifugal force of from about 50 to about 300;
(d) serially diluting the Proteinase K-treated sample of viral genome with the dilution buffer and mixing the serially diluted sample of viral genome with a relative centrifugal force of from about 50 to about 300;
(e) preparing a master mix comprising a primer-probe mix for the ddPCR assay and mixing the prepared master mix at a relative centrifugal force of from about 50 to about 300; and
(f) loading the serially diluted sample of viral genome with the master mix comprising the primer-probe mix on a ddPCR plate for the ddPCR assay.

2. The method of claim 1, wherein the sample of viral particles comprises adeno-associated virus (AAV) particles.

3. The method of claim 2, wherein the AAV particles are of serotype AAV1, AAV2, AAV3, AAV4, AAV5, AAV6, AAV7, AAV8, AAV9, AAV-DJ, AAV-DJ/8, AAV-Rh10, AAV-retro, AAV-PHP.B, AAV8-PHP.eB, or AAV-PHP.S.

4. The method of claim 1, wherein the gentle mixing is conducted using a vortex mixer with a relative centrifugal force of from about 20 to about 100, or of from about 40 to about 60.

5. The method of claim 1, wherein the gentle mixing is performed for a period of from about 10 seconds to about 45 seconds.

6. The method of claim 1, wherein the mixing is carried out using a vortex mixer with a relative centrifugal force of from about 100 to about 200, or of from about 110 to about 140.

7. The method of claim 1, wherein the mixing is performed for a period of from about 10 seconds to about 45 seconds.

8. The method of claim 1, wherein the mixing, gentle mixing, or both the mixing and the gentle mixing is performed for a period of from about 10 seconds to about 20 seconds.

9. The method of claim 1, wherein the serially diluted sample of viral genome is mixed with the master mix comprising the primer-probe mix at a relative centrifugal force of from about 50 to about 300 before loading on the ddPCR plate as well as after sealing of the plate.

10. (canceled)

11. The method of claim 1, wherein the dilution buffer comprises a non-ionic surfactant and a sheared salmon sperm DNA, and wherein the dilution buffer is filtered through a 0.20-0.25 μm syringe filter.

12. The method of claim 1, wherein the primer-probe mix comprises at least one oligonucleotide probe that is detectably labeled and at least two oligonucleotide primers targeting a gene of interest.

13. The method of claim 12, wherein the oligonucleotide probe comprises a nucleotide sequence of SEQ ID NO: 3.

14. The method of claim 12, wherein the oligonucleotide primers further comprise a forward primer with a nucleotide sequence of SEQ ID NO: 1 and a reverse primer with a nucleotide sequence of SEQ ID NO: 2.

15. A method of performing a droplet digital PCR (ddPCR) assay on a sample of adeno-associated virus (AAV) genome prepared according to the method of claim 1, the method comprising:
(a) performing the ddPCR assay on the sample of AAV genome loaded in the ddPCR plate with the ddPCR master mix;
(b) analyzing data obtained from the ddPCR assay; and
(c) determining a viral titer based on analysis of the data obtained from the ddPCR assay.

16. The method of claim 15, wherein the adeno-associated virus is of serotype AAV1, AAV5, or AAV8.

17. The method of claim 15, wherein a threshold amplitude for analysis of the data is set at 6000.

18. The method of claim 15,
wherein analysis of the data includes data with a copy number between 200 copies/μL and 8000 copies/μL.

19. The method of claim 15, wherein the sample of AAV genome comprises an exogenous gene.

20. The method of claim 19, wherein the exogenous gene is a therapeutic gene; and/or wherein determining the viral titer includes determining the viral titer of the exogenous gene in viral genomes/mL based on analysis of the data obtained from the ddPCR assay.

21. (canceled)

22. The method of claim 15, wherein the ddPCR assay produces a coefficient of variation of no more than 5% or a coefficient of variation of less than 2%.

23. (canceled)
